(19) United States
(12) Patent Application Publication
(10) Pub. No.: US 2022/0175544 A1
(43) Pub. Date: Jun. 9, 2022

(54) TRI-FLANGE HUMERAL PROSTHESIS

(71) Applicant: Shoulder Innovations, Inc., Holland, MI (US)

(72) Inventors: Robert J. Ball, Holland, MI (US); Donald E. Running, Holland, MI (US)

(21) Appl. No.: 17/652,046

(22) Filed: Feb. 22, 2022

Related U.S. Application Data
(63) Continuation of application No. 16/980,335, filed on Sep. 11, 2020.
(60) Provisional application No. 62/641,990, filed on Mar. 12, 2018.

(51) Int. Cl. A61F 2/40

(52) U.S. Cl. CPC A61F 2/4081; A61F 2/4014; A61F 2002/4085; A61F 2002/4051; A61F 2/4059

(57) ABSTRACT

In some embodiments, disclosed is a glenohumeral implant with improved joint mobility. The implant can include a backing component; a neutral non-inclined bearing component on a concave side of the backing component, the bearing component made from a different material than the backing component and configured to touch a glenosphere having a center of rotation.

### PRIORITY CLAIM

This application claims the benefit under 35 U.S.C. § 119(e) as a nonprovisional application of U.S. App. No. 62/641,990 filed on Mar. 12, 2018, which is hereby incorporated by reference in its entirety.

### BACKGROUND

Shoulder Replacement is a commonly performed medical procedure for treatment of osteoarthritis, rheumatoid arthritis, as well as for treatment of certain deformities related to oncological indications as well as trauma. There are two primary types of articulations available to surgeons for treatment: anatomic and reverse. With anatomic, the surgeon replaces the articular surfaces with industrial materials such that the articulating surfaces are substantially the same shape as the natural anatomy. A stem can be commonly fixed inside the canal of the humerus, a metallic articular head can be rigidly fixed to the proximal aspect of the same, the articular head having a convex articular surface adapted to articulate with the glenoid implant. The glenoid implant can include on its back side (medial side) certain pegs or posts or fins adapted to be rigidly fixed within the glenoid fossa of the scapula and on its front side a concave or flat articular surface adapted to articulate with the humeral head of the humeral implant.

When a reverse prosthesis is used, the articular surface is reversed in that the metallic ball is rigidly fixed to the glenoid fossa of the scapula, and the concave articular surface is rigidly fixed to the humeral bone, thereby reversing the fashion of articulation of the prosthesis.

The surgeon chooses between the two types of prostheses by assessing a number of conditions of the patient including level of pain, patient activity level, deformity or severity of the boney degradation, the strength of surrounding soft tissues, and present or absence of prior surgery, and particularly the health and strength of the rotator cuff muscle and tendon. Disease of the rotator cuff is common among patients with arthritis of the shoulder. In this circumstance, it is commonly observed that the absence of insufficiency of the rotator cuff leads to a condition where the anatomic shoulder replacement prosthesis is not sufficiently stabilized by surrounding soft tissue. In this case, a reverse shoulder replacement prosthesis can be preferred in some cases due to the higher inherent stability of the articulation. In addition, the reverse prosthesis can advantageously utilize the remaining muscles in a way they can be more effective in the absence of the other soft tissue structures by adjusting the position of the articular surfaces within the joint.

It is not uncommon that a surgeon selects to use an anatomic prosthesis and is provides effective treatment to the patient though the shoulder replacement operation. However, over time and during use of the prosthesis, the patient's rotator cuff complex can become insufficient, tear, or generally be diseased such that it can no longer perform its function associated with normal joint kinematics. In this case, the surgeon can elect to perform a second operation to remove the anatomic prosthesis, and replace the anatomic prosthesis with a reverse prosthesis.

Several attempts have been made to attempt to address the need of conversion of the articular surface without interruption of the fixation. Primarily, these are created using a two (or more) system, where there is a metallic fixation component which is rigidly fixed to the glenoid fossa, and a polyethylene (PE) articular component which is secondarily fixed to the metallic component, and provides the concave articular surface adapted to articular with the humeral prosthesis. While referred to herein as a PE component, some embodiments do not require the use of polyethylene and can be made of other biocompatible materials depending on the desired clinical result. The PE component is commonly fixed to the metallic fixation component by conventional industrial techniques such as snap fit mechanisms, snap rings, compression pins, overmolding of the PE and other such means.

A challenge of this particular articulation in some cases is that the glenoid fossa is relatively small, and commonly there is much reduced presence of bone in patients with arthritis. In this context, the surgeon has limited positioning and bone to work with in order to fit within the patient. In addition, the surgeon must be careful not to overstuff the joint, meaning implant components that move the new articulating surface far from its original position such that the soft tissues is unnaturally tensioned, which can lead to instability, accelerated where, soft tissue failure, pain, reduced range of motion, or fracture of the prosthesis and surrounding bone. Facing these conditions, the prosthesis typically needs to be designed to remain relatively thin (commonly, 1 piece, where PE glenoid implants typically have a 4 mm thick articular surface). In order to design these modular components, there can be little additional packaging space provided into which to fit the attachment mechanisms necessary for use without adversely affecting the performance of the overall joint replacement procedure. Thus, typically, these designs lead to “over-optimization” of the fixation and articular portions in order to provide sufficient attachment mechanisms such that either: the PE is too thin to be sufficiently strong, the metallic components are too small to provide sufficient fixation, or the overall mechanism is insufficiently rigid causing there to be secondary wear surfaces, and generation of wear particles leading to PE disease.

A problem that can exist is that in the case where the surgeon wants to change the prosthesis type, the anatomic prosthesis is commonly well fixed and adapted to the patient's body such that removal of the prosthesis can be very destructive, and leave natural bone remaining that is perhaps insufficient to support the fixation of the reverse prosthesis. What is needed is a prosthesis system that provides a means by which the articulating surfaces of the implant can be exchanged such that the anatomic surfaces can be converted to reverse surfaces, while not exchanging the fixation components.

What is also needed is a simple means by which the surgeon can implant an inset anatomic articulating glenoid implant whereby at a later date, can remove the anatomic articulating surface and replace it with a reverse articulating surface such that the primary means of fixation remains well fixed in the glenoid fossa at the moment of articular exchange.

Furthermore, conventional systems can change the inclination angle of the humerus by offering bearing components (e.g., polyethylene inserts) that are more and more inclined, thus medializing the center of rotation of the glenosphere and overstuffing the joint. Systems that can change the inclination angle of the humerus without changing the center of rotation of the glenosphere and/or overstuffing the joint to provide increased range of motion are needed.

### SUMMARY

In some embodiments, disclosed is a glenohumeral implant with improved joint mobility. The implant can include a backing component; a neutral non-inclined bearing component on a concave side of the backing component, the bearing component made from a different material than the backing component and configured to touch a glenosphere having a center of rotation, wherein the bearing component is substantially radially symmetrical; and a peg extending from the backing component. The longitudinal axis of the peg of the backing component can be offset from the center of rotation of the glenosphere, while the central axis of the backing component (e.g., tray) can be aligned with the center of rotation of the glenosphere. The backing component can be inclined, for example, by an angle of between about 1.25 degrees and about 30 degrees, or between about 2.5 degrees and about 12.5 degrees with respect to a corresponding attachment surface of a humeral head. The glenoid implant when inserted into a patient does not change the center of rotation of the glenosphere in some embodiments, and can be convertible from an anatomic to a reverse prosthesis. In some embodiments, the bearing component has a depth of less than about 5.5 mm, such as between about 3 mm and about 5 mm in some cases. The bearing component can include a plastic material, such as polyethylene. The backing component can include a metal material. The peg can have a Morse or other taper. The peg can be offset along the long axis of the backing component.

Also disclosed herein is a method of implanting a glenoid implant, comprising: reaming a cavity in the glenoid surface; sizing a first glenoid implant comprising a first angulation with respect to a stem, wherein the glenoid implant comprises a neutral bearing component and an inclined backing component comprising a first incline angle with respect to a corresponding attachment surface of a humeral head; sizing a second glenoid implant comprising a second angulation with respect to the stem, wherein the glenoid implant comprises a neutral bearing component and an inclined backing component comprising a second incline angle with respect to a corresponding attachment surface of a humeral head; implanting the second glenoid implant in the glenoid cavity; wherein the center of rotation of the glenosphere does not change between the first glenoid implant and the second glenoid implant, allowing for adjustment of version and inclination without changing the center of rotation.

Some embodiments of the invention are focused on advantageously exchanging the articular surface of the glenoid from a concave shape to a convex shape, without removing the components or the interface having to do with fixation of the implant into the glenoid fossa, by utilizing a convertible humeral prosthesis.

In some embodiments, embodiments of the invention can be used or modified with use with particular advantages of using inset glenoid fixation technology in anatomic shoulder arthroplasty, such as described, for example, in U.S. Pat. No. 8,007,538 to Gunther, which is hereby incorporated by reference in its entirety.

In some embodiments, embodiments of the invention can be used as a modular platform for anatomic or reverse articulations.

In some embodiments, embodiments of the invention can be used to change the inclination angle of the prosthetic with respect to the humerus.

In some embodiments, a glenoid implant allowing for adjustable angles for anatomic or reverse arthroplasty is presented and comprises: a backing component; a bearing component on the concave side of the backing component, the bearing component made from a different material than the backing component; and a peg extending from the backing component. Further, in some embodiments the longitudinal axis of the backing component is offset from the center of rotation, but the center of rotation is not changed.

In some embodiments, the embodiments of the bearing component lining the backing component is made of an evenly distributed biologically compatible material.

In some embodiments, the bearing component is made of a material such as polyethylene.

In some of the embodiments, embodiments of the invention offer different angular placements, allowing for a range of inclination angles.

In some embodiments, disclosed herein is a shoulder implant with improved joint mobility. The implant can include a backing component; a neutral non-inclined bearing component on a concave side of the backing component, the bearing component made from a different material than the backing component and configured to touch a glenosphere having a center of rotation, wherein the bearing component is substantially radially symmetrical; and a peg extending from the backing component and configured to be connected to a humeral stem. The longitudinal axis of the peg of the backing component can be offset from the center of rotation of the glenosphere. The longitudinal axis of the backing component can be aligned with the center of rotation of the glenosphere.

In some embodiments, the backing component is inclined by an angle of between about 1.25 degrees and about 30 degrees with respect to a corresponding attachment surface of a humeral head or stem.

In some embodiments, the backing component is inclined by an angle of between about 2.5 degrees and about 12.5 degrees with respect to a corresponding attachment surface of a humeral head or stem.

In some embodiments, the implant when inserted into a patient does not change the center of rotation of the glenosphere.

In some embodiments, the implant is configured for anatomic positioning, or for reverse positioning.

In some embodiments, the bearing component has a depth of less than about 5.5 mm, or between about 3 mm and about 5 mm.

In some embodiments, the bearing component comprises a plastic material, such as polyethylene.

In some embodiments, the backing component comprises a metal material.

In some embodiments, the peg comprises a Morse taper.

In some embodiments, at least a portion of an inferior-facing surface of the backing component is configured to come in contact with a prepared humeral surface comprises an oval or elliptical cross-section, at least a portion of the inferior-facing surface of the bearing component comprises a circular cross-section, and at least a portion of the superior-facing surface of the backing component comprises a circular cross-section, the portion of the inferior-facing surface of the bearing component and the superior-facing surface of the backing component configured to snap or otherwise mate with each other.

In some embodiments, disclosed is a kit of shoulder implants, comprising a plurality of implants as in claim 1, wherein the plurality of implants comprises a first implant and a second implant, wherein the backing component of the first implant is inclined by a first angle, wherein the backing component of the second implant is inclined by a second angle different from the first angle, wherein the center of rotation of the glenosphere of the first implant and the second implant are in the same location.

Also disclosed herein is a method of implanting a shoulder implant. The method can include reaming a cavity in the glenoid surface; sizing a first implant comprising a first angulation with respect to a stem, wherein the implant comprises a neutral bearing component and an inclined backing component comprising a first incline angle with respect to a corresponding attachment surface of a humeral head; and sizing a second glenoid implant comprising a second angulation with respect to the stem. The glenoid implant can comprise a neutral bearing component and an inclined backing component comprising a second incline angle with respect to a corresponding attachment surface of a humeral head. The method can also include implanting the second glenoid implant in the glenoid cavity. The center of rotation of the glenosphere does not change between the first glenoid implant and the second glenoid implant, allowing for adjustment of version and inclination without changing the center of rotation.

In some embodiments, also disclosed herein is a humeral prosthesis, comprising a proximal end comprising at least three radially outwardly extending fins; and a stem extending distally from the proximal end to a distal end. The proximal end can include a proximal end diameter defined by a circle contacting radial outward-most proximal tips of the at least three radially outwardly extending fins. The distal end can include a distal end diameter. The proximal end diameter can be, for example, between about 30 mm and about 50 mm. The distal end diameter can be between, for example, between about 5 mm and about 6 mm. A ratio of the proximal end diameter and the distal end diameter can be between about 5 and about 9, or between about 5.5 and about 8.5. The proximal end can include a bowl-shaped concavity. The prosthesis can also include a central aperture in the proximal end configured to mate with a reverse component.

### BRIEF DESCRIPTION OF THE DRAWINGS

FIG. 1 is a side perspective view of an embodiment of a convertible humeral prosthesis, according to some embodiments of the invention.

FIG. 2 is a bottom perspective view of the embodiment shown in FIG. 1;

FIG. 3 is a top view of the embodiment shown in FIG. 1 also showing an aperture in the center of the superior facing surface of the neck of the prosthesis and configured to be utilized as a reverse adapter for, in some cases, facilitating placement or removal of a spherical ball on the humeral head.

FIG. 4 is a side view of the embodiment shown in FIG. 1;

FIG. 5 is another side view of the embodiment shown in FIG. 1, also illustrating channel of the reverse adapter aperture extending within the neck and stem.

FIG. 6 is an inverted perspective view, also illustrating medial groove along the neck and stem of the prosthesis.

FIG. 7 is another perspective view of the prosthesis shown in FIG. 1.

FIG. 7A is a side perspective view of another embodiment of a convertible humeral prosthesis, according to some embodiments of the invention.

FIG. 7B is a bottom perspective view of the embodiment shown in FIG. 7A;

FIG. 7C is a top view of the embodiment shown in FIG. 7A also showing an aperture in the center of the superior facing surface of the neck of the prosthesis and configured to be utilized as a reverse adapter for, in some cases, facilitating placement or removal of a spherical ball on the humeral head.

FIG. 7D is a side view of the embodiment shown in FIG. 7A;

FIG. 7E is another side view of the embodiment shown in FIG. 7A, also illustrating channel of the reverse adapter aperture extending within the neck and stem.

FIG. 7F is an inverted perspective view, also illustrating medial groove along the neck and stem of the prosthesis.

FIG. 7G is another perspective view of the prosthesis shown in FIG. 7F.

FIGS. 8-11 illustrate various views of another embodiment of a humeral replacement prosthesis.

FIG. 12 is an illustration of one potential embodiment of the invention that depicts a conventional glenoid implant with an offset bearing component.

FIG. 13 is an illustration of one embodiment of the offset backing component allowing a 2.5 degree inclination.

FIG. 14 is an illustration of one embodiment of the offset backing component allowing a 7.5 degree inclination.

FIG. 15 illustrate the use of one embodiment of the offset backing component allowing a 12.5 degree inclination.

### DETAILED DESCRIPTION

In particular, some embodiments of the invention are focused on advantageously exchanging the articular surface of the glenoid from a concave shape to a convex shape, without removing the components or interface having to do with fixation of the implant into the glenoid fossa, by utilizing a convertible humeral prosthesis.

In some embodiments, embodiments of the invention can be used or modified with use with particular advantages of using inset glenoid fixation technology in anatomic shoulder arthroplasty, such as described, for example, in U.S. Pat. No. 8,007,538 to Gunther, which is hereby incorporated by reference in its entirety. In some embodiments, an inset method includes identifying a patient having a glenoid surface; reaming a cavity into the glenoid surface; and inserting a glenoid implant having a body and a single, radially symmetric central peg oriented along a central axis of the implant, the body having a bearing surface on a peripheral edge thereof into the cavity, such that at least a portion of a peripheral edge of the body is inset with respect to the cavity and resides below the adjacent glenoid surface and the portion residing below the adjacent glenoid surface is circumferentially surrounded by cortical bone of the glenoid.

What is further described are methods by which the surgeon can achieve the use of the inset glenoid technology with an anatomic articulation, while after having the ability to convert the technology to a reverse articulation, without requiring removal the rigid fixation between the inset fixation and the scapula bone (in other words, allowing the rigid fixation support between the inset fixation and the scapula bone to remain in place during conversion from an anatomic to a reverse prosthesis).

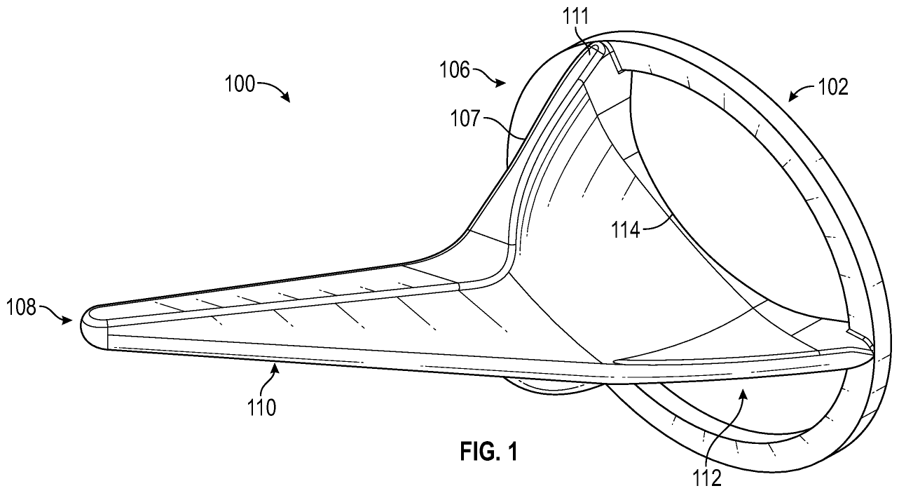

FIGS. 1-7 illustrate various views of a humeral replacement prosthesis 100, according to some embodiments of the invention. The prosthesis 100 can optionally include a proximal ring element 102, such as a collar (which can be configured to be attached to a spherical ball head portion, not shown) with a peripheral edge 103 that can be annular such as circular as shown, with an inferior-facing surface that can be optionally recessed (including a convex or flat bowl-like shape) and include a cavity, or inline in other embodiments. The humeral prosthesis 100 can include a superior-facing surface 114 of the proximal end 106, and be concave and bowl-shaped as shown with the base and deepest portion of the bowl proximate the center of the superior facing surface 114 of the proximal end 106 such that there is a space 113 between the inferior-facing surface of the humeral head portion (not shown) and the superior-facing surface 114 of the proximal end 106 of the prosthesis 100. In some embodiments the depth of the bowl at the center of concave curvature is between about 3 mm and about 7 mm, such as between about 4 mm and about 6 mm deep, such as about 5 mm deep. The proximal end 106 of the prosthesis 100 can include spaced-apart flanges 107, such as three flanges 107 spaced equally apart, with vertices 111 of the flanges 107 extending radially outwardly while becoming narrower from the center of the proximal end 106 of the prosthesis 100. In some cases, a tri-flange design can provide for improved stability and rotation prevention, among other advantages. Extending distally from the proximal end 106 at an angle to the longitudinal axis of the proximal end/neck 106 is the stem 110 and distal end 108 of the prosthesis 100.

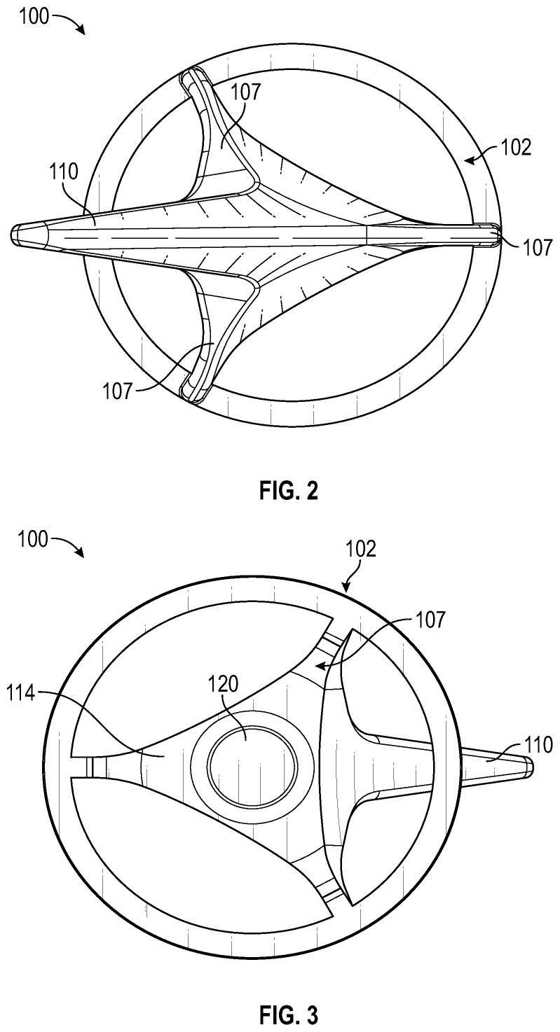

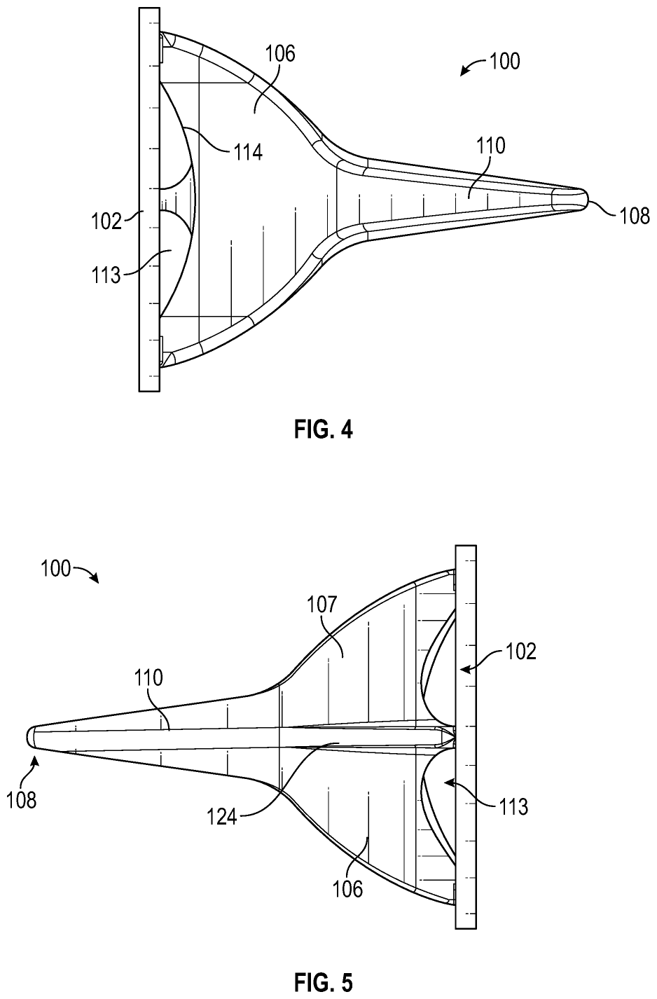

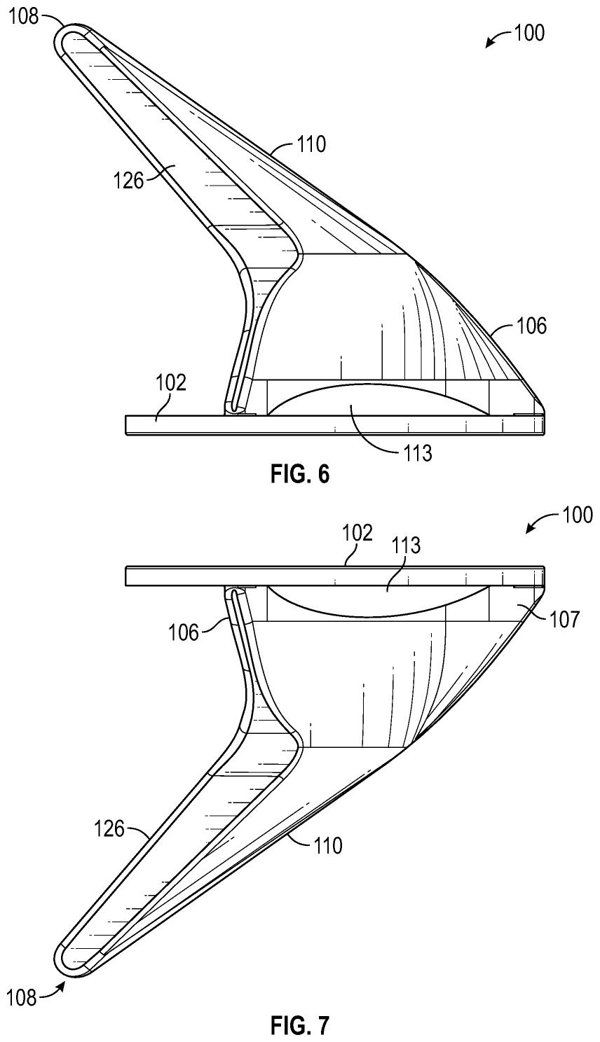

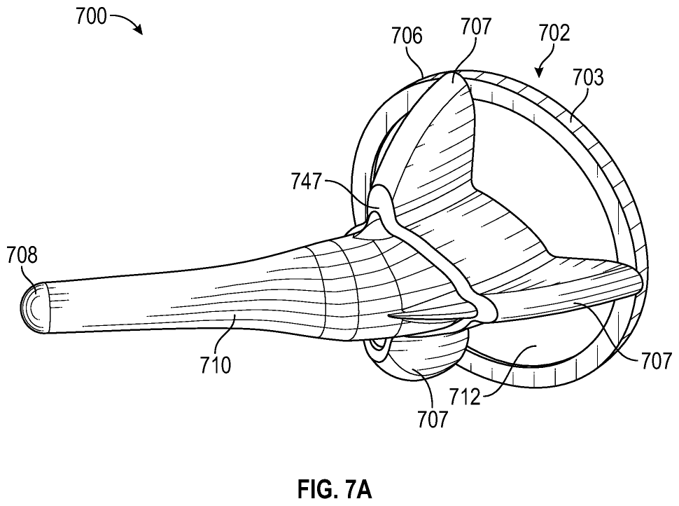

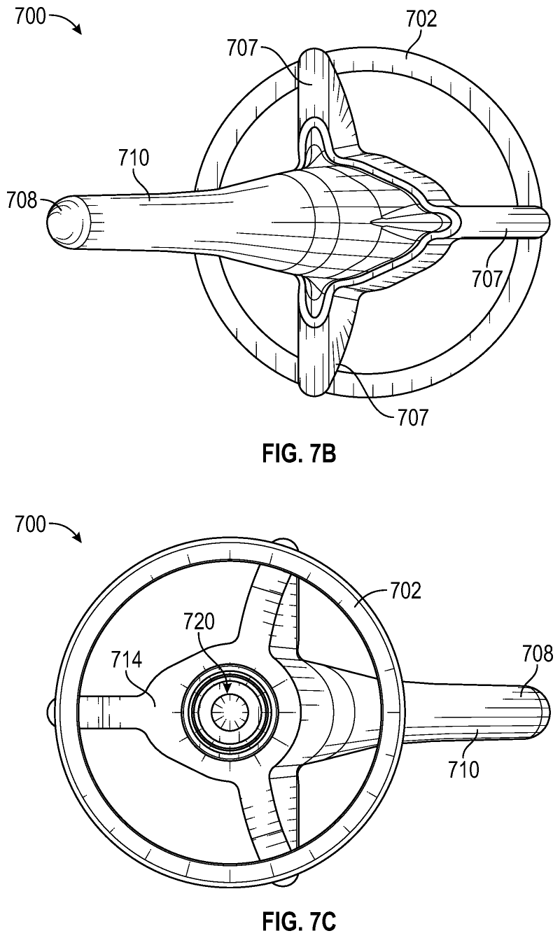

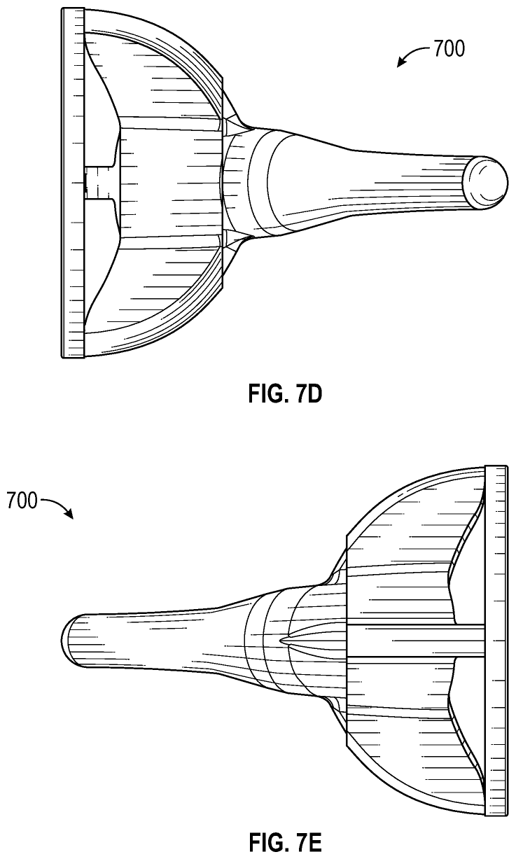

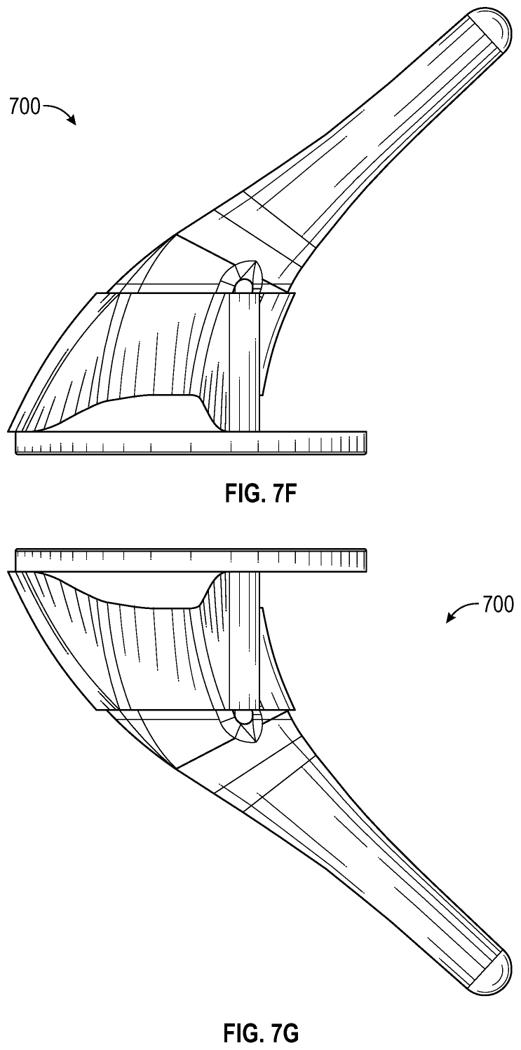

In some embodiments, the stem 110 and distal end 108 of the prosthesis has reduced thickness, e.g., tapering to no more than about 6 mm, 5.5 mm, 5 mm, 4.5 mm, 4 mm, or less in width dimension, such as between about 5.5 mm and about 6 mm, and ranges incorporating any two of the foregoing values, to advantageously reduce the quantity of native humerus bone needed to be removed, and allows for a stem 110 that is 3-4 mm or longer than a conventional stem 100 and allows for the majority of the fixation to occur in the proximal portion (e.g., neck 106) of the prosthesis. FIG. 1 is a side perspective view of an embodiment of the prosthesis; FIG. 2 is a bottom perspective view of the embodiment shown in FIG. 1; FIG. 3 is a top view of the embodiment shown in FIG. 1 also showing an aperture 120 in the center of the superior facing surface 114 of the neck 106 of the prosthesis and configured to be utilized as a reverse adapter for, in some cases, facilitating placement or removal of a spherical ball on the humeral head. FIG. 4 is a side view of the embodiment shown in FIG. 1; FIG. 5 is another side view of the embodiment shown in FIG. 1, also illustrating channel 124 of the reverse adapter aperture 120 extending within the neck 106 and stem 110. FIG. 6 is an inverted perspective view, also illustrating medial groove 126 along the neck 106 and stem 110 of the prosthesis 100. FIG. 7 is another perspective view of the prosthesis shown in FIG. 1.

FIGS. 7A-7G illustrate views of another embodiment of a humeral replacement prostheses somewhat similar to, and which can incorporate any number of features shown, for example in FIGS. 1-7. FIG. 7A illustrates a perspective view of the prosthesis 700 including optional proximal ring element 702, such as a collar, with a peripheral edge 703 that can be annular such as circular as shown, with an inferior-facing surface that can be optionally recessed (including a convex or flat bowl-like shape) and include a cavity, or inline in other embodiments. The humeral prosthesis 700 can include a superior-facing surface 714 of the proximal end 706, and be concave and bowl-shaped as shown with the base and deepest portion of the bowl proximate the center of the superior facing surface 714 of the proximal end 106 such that there is a space 713 between the inferior-facing surface of the humeral head portion (not shown) and the superior-facing surface 714 of the proximal end 706 of the prosthesis 700. The proximal end 706 of the prosthesis 700 can include spaced-apart flanges 707, such as three flanges 707 spaced equally apart, with vertices 711 of the flanges 707 extending radially outwardly while becoming narrower from the center of the proximal end 706 of the prosthesis 700. Extending distally from the proximal end 106 at an angle to the longitudinal axis of the proximal end/neck 706 is the stem 710 and distal end 708 of the prosthesis 700. Also illustrated is transition zone 747, of which proximal to that zone 747 the prosthesis 700 includes a porous coating, and distal to that zone 747 the prosthesis 700 does not include a porous coating. The coating could include, for example, a plasma spray, porous metal, hydroxyapatite, or other component which can facilitate cementless fixation to bone. However, cement fixation can be utilized in some embodiments.

FIG. 7A is a side perspective view of an embodiment of the prosthesis; FIG. 7B is a bottom perspective view of the embodiment shown in FIG. 7A; FIG. 7C is a top view of the embodiment shown in FIG. 7A also showing an aperture 120 in the center of the superior facing surface 114 of the neck 106 of the prosthesis and configured to be utilized as a reverse adapter for, in some cases, facilitating placement or removal of a spherical ball on the humeral head. FIG. 7D is a side view of the embodiment shown in FIG. 7A; FIG. 7E is another side view of the embodiment shown in FIG. 7A, also illustrating channel 124 of the reverse adapter aperture 120 extending within the neck 106 and stem 110. FIG. 7F is an inverted perspective view, also illustrating medial groove 126 along the neck 106 and stem 110 of the prosthesis 100. FIG. 7G is another perspective view of the prosthesis shown in FIG. 7.

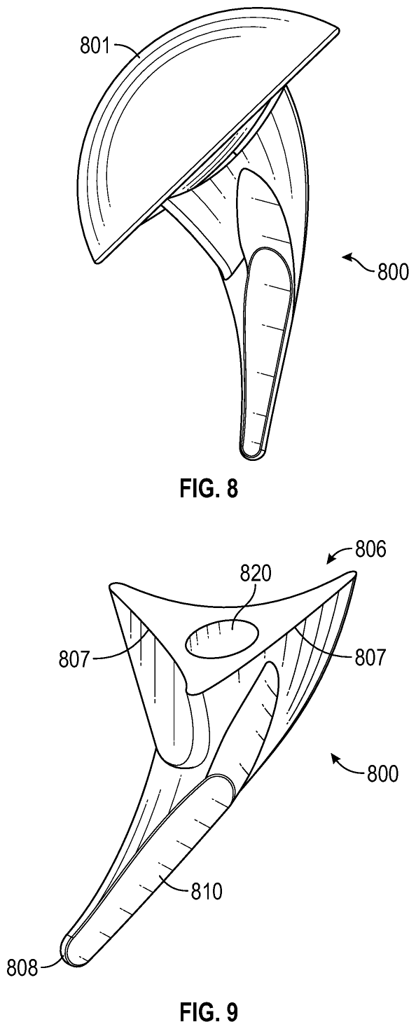

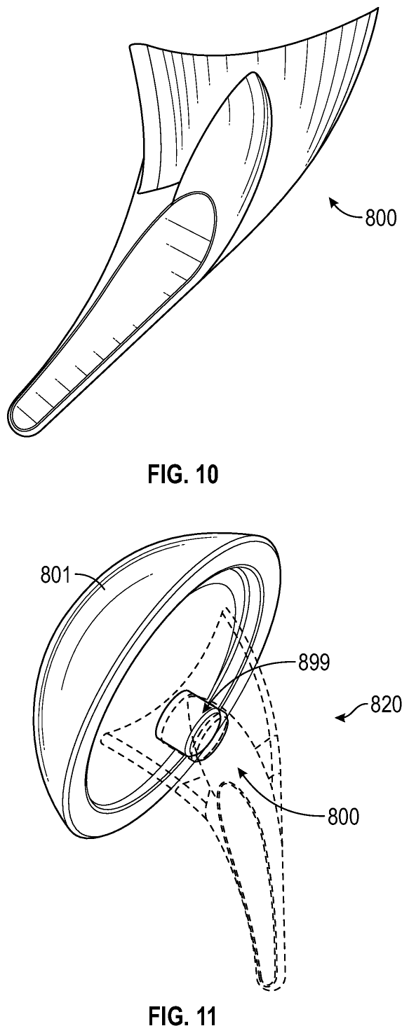

FIGS. 8-11 illustrate various views of another embodiment of a humeral replacement prosthesis. FIG. 8 illustrates a humeral prosthesis that can be as described elsewhere herein, configured to be connected to a humeral head 801. FIG. 9 illustrates a perspective view of the humeral prosthesis 800, including stem 806, flanges 807, and adapter aperture 820 on the proximal end 806, as well as distal end 808. FIG. 10 illustrates a side view of the humeral prosthesis 800. FIG. 11 illustrates humeral head prosthesis 801 configured to be connected via projection 899 to the adapter aperture 820 of the humeral prosthesis 800, such as via press fit, threads, adhesive, or other techniques.

As shown, some potentially advantages of humeral implant configurations as described herein include that fixation is concentrated proximally, to conserve native bone. The prosthesis could be cemented, or cementless in some embodiments. The prosthesis geometry allows for ease of use, minimized surgical steps, and robust as errors can be readily absorbed. The prosthesis can also be convertible to reverse, which increases potentially for success and prevents or minimizing overstuffing. The prosthesis can also be advantageously inexpensive, logistically simple, and have minimal inventory requirements, as a diameter at or near the distal end of the stem can be constant, while the diameter at or near the proximal end of the stem can vary. For example, an operator could select a small number of humeral stem implants, e.g., with 34 mm, 38 mm, and 44 mm proximal diameters (which can be measured as the smallest circle that can encircle the proximal-most radially-outward end elements of the fins), all of which have the same distal-most diameter, such as about 5.5 mm or about 6 mm, or no more than about 7 mm, 6.5 mm, 6 mm, 5.5 mm, 5 mm, or less, or ranges including any two of the foregoing values. In some embodiments, the proximal-most diameter can be between about 25 mm and about 50 mm, such as about 25, 30, 35, 40, 45, 50 mm, or ranges including any two of the foregoing values. In some embodiments, the humeral stem implants can have a ratio of proximal-most to distal-most diameter of the humeral stem of between about 5 and about 9, between about 5.5 and about 8.5, about 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, or ranges including any two of the foregoing values. The ability to provide a set or array of humeral stem implants that a physician can choose from with varying proximal-most diameters, each with the same or substantially the same small distal-most diameter can be in contrast to conventional humeral stem prostheses, in which within a set of different humeral stem sizes, the distal-most diameter generally also increases as the proximal-most diameter increases. In some embodiments, the prosthesis is collarless. In some embodiments, the prosthesis has a total length of about or at least about 50 mm, 51 mm, 52 mm, 53 mm, 54 mm, 55 mm, 56 mm, 57 mm, 58 mm, 59 mm, 60 mm, 65 mm, 70 mm, or more, or ranges incorporating any two of the aforementioned values.

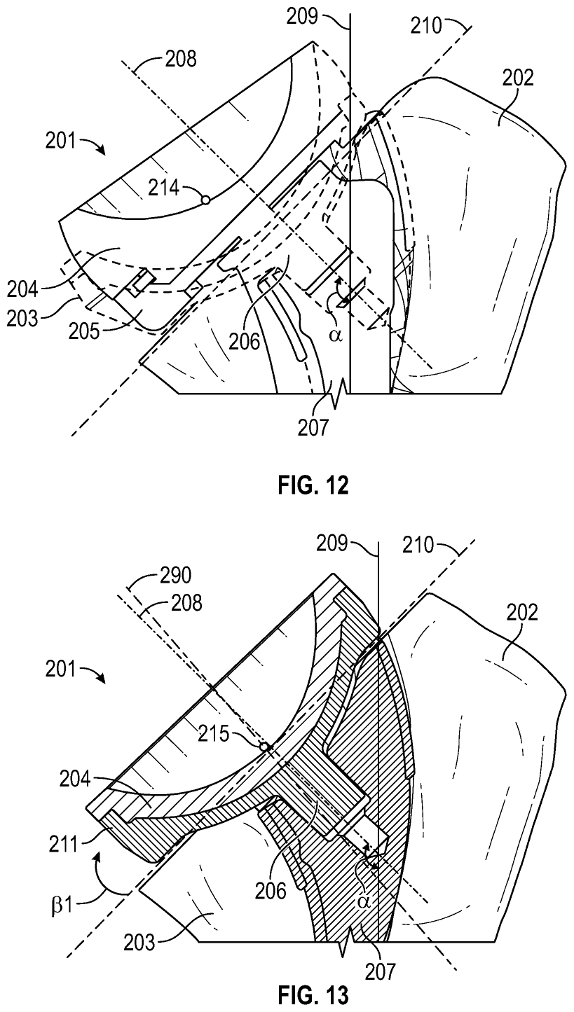

FIGS. 12-15 illustrate various views of several embodiments of components of a glenohumeral replacement prosthesis. FIG. 12 can be used to compare the effects of several different embodiments of the invention as depicted in FIGS. 13-15.

FIG. 12 depicts a conventional shoulder implant component embedded into the fossa of the scapula 201, such as the glenoid, or a humerus for example. FIG. 12, depicts an implant with an inclined, offset bearing component 204 (referring to the central axis of the bearing component being offset from the central axis of the backing component), with a backing component 205 which is aligned with center of rotation 208. The prosthetic is embedded on or within the bony cavity 203 comprises a bearing surface 204 embedded onto a backing component (e.g., a concave tray) 205. The bearing surface 204 lines the backing component 205 and can be made of any appropriate material, such as a non-metallic, biologically compatible material (e.g. polyethylene). The backing component connects to an elongated stem 207 that is embedded into the bone 202. The backing component 205 connects with the elongated stem 207 by a peg or keel (e.g. a morse taper) 206. In FIG. 12, the stem has neck shaft angle of about 132.5 degrees α which is the angle created by intersection of the axis through the elongated stem 209 and the center of rotation 208. The backing component 205 is aligned with the axis of the fossa 210. Since the bearing component 204 is offset, the center of rotation 208 of the glenosphere 214 is medialized.

FIG. 13 illustrates a different embodiment wherein the inclination angle of the humerus angle is controllable exclusively by an offset backing component 211, while the bearing component is not offset. By offset, the peg 206 of the backing component 211 is positioned such that a first distance from the peg 206 to a first end of the offset backing component 211 is not equal to a second distance from the peg 206 to a second send of the offset backing component 211. In some embodiments, the first distance is at least about, about, or no more than about 5%, 10%, 15%, 20%, 25%, or more or less than the second distance, or ranges including any two of the foregoing values. This is in distinct contrast to FIG. 12 wherein the bearing component (e.g., polyethylene) is offset with respect to a central axis of the backing component. FIG. 13 shows the effect of a reverse articulation at 135 degrees using a backing component that provides a 2.5 degree incline 211 when attached to the elongated stem 207. The rotation β1 represents a rotation of 2.5 degrees about the axis of the glenoid fossa 210. When added to the angle α, a reverse articulation of 135 degrees is achieved. Since the bearing component is not offset as in FIG. 12, the center of rotation of the glenosphere 215 remains constant, and is not medialized (or lateralized) away from the center of rotation 208. Also, the central axis 290 of the backing component 211 is offset from the central axis 208 of the peg.

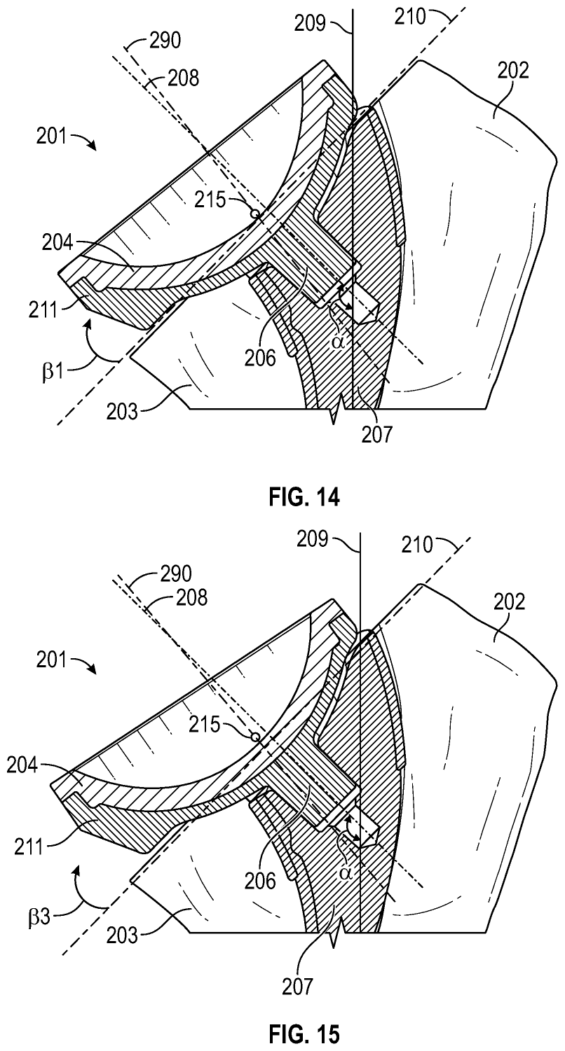

FIG. 14 is similar to FIG. 13 with the exception that the angle of inclination of the backing component is different. FIG. 14 shows a desired 140 degree angle with a backing component providing a 7.5 degree incline 212 when attached to the elongated stem 207. The rotation β2 represents a rotation of 7.5 degrees about the axis of the glenoid fossa 210. When added to the angle α, a reverse articulation of 140 degrees is achieved. Since the bearing component is not offset as in FIG. 12, the center of rotation of the glenosphere 215 is not medialized away from the center of rotation 208 and remains in the same position as it was in FIG. 13.

FIG. 15 illustrates a 145 degree angle with a backing component providing a 12.5 degree incline 213 when attached to the elongated stem 207. The rotation β3 represents a rotation of 12.5 degrees about the axis of the glenoid fossa 210. When added to the angle α, a reverse articulation of 145 degrees is achieved. Since the bearing component is not offset as in FIG. 12, the center of rotation of the glenosphere 215 is not medialized away from the center of rotation 208 and remains in the same position as it was in FIG. 13 and FIG. 14.

In some embodiments of the invention, the inclination angles produced by the backing component above and beyond the neck shaft angle of the elongated stem may range from about 1.25, 2.5, 3.75, 5, 7.5, 10, 12.5, 15, 17.5, 20, 22.5, 25, 27.5, 30, 32.5, 35 degrees, or ranges including any two of the aforementioned values.

The native prepared surface of the humerus of which an inferior-facing surface of the backing component rests against after implementation generally has an oval or elliptical cross-section. In some embodiments, the inferior-facing surface of the backing component also has an oval or elliptical cross-section substantially matching that of the surface of the humeral bone. In some embodiments, at least a portion of the inferior facing surface of the bearing component (e.g., polyethylene component in some cases) has a circular cross-section, and is configured to snap into, or otherwise mate with a complementary portion of the superior-facing surface of the backing component, which can also have a circular cross-section.

Non-limiting potential advantages of the embodiments in FIG. 13-15 is observed by example when comparing FIG. 12 to FIG. 14. The depth of the backing component in FIGS. 13-15 can be about 5 mm, compared with about 7.5 mm with respect to the FIG. 12 embodiment where the bearing component is offset with respect to the center of rotation of the glenosphere, as well as the central axis of the backing component. In some embodiments, the depth of the backing component is less than about 6, 5.5, 5, 4.5, 4, 3.5, 3 mm or less, or ranges including any two of the aforementioned values. Changing the backing component angle in FIGS. 13-15, as opposed to changing the amount of polyethylene plastic or other material in the bearing component, results in an additional amount of space, such as about 2.5 mm or more or less of space formed in the bowl for rotation of the glenosphere. Therefore, in several of the embodiments presented, the joint is less “stuffed” and not overstuffed and can potentially have greater range of motion. Furthermore, a physician can select between a range of different backing components with varying inclination angles, while the bearing components are neutral (e.g., not inclined). As such, the inclination angle of the humerus can be controlled solely by selecting an appropriate backing component, and the center of rotation of the glenosphere remains constant irrespective of the potentially varying inclination angle of the backing component.

In some embodiments, a glenohumeral implant allowing for adjustable angles for anatomic or reverse arthroplasty is presented and comprises: a backing component; a bearing component on the concave side of the backing component, the bearing component made from a different material than the backing component; and a peg extending from the backing component. Further, in some embodiments the longitudinal axis of the backing component is offset from the center of rotation, but the center of rotation is not changed.

Various other modifications, adaptations, and alternative designs are of course possible in light of the above teachings. Therefore, it should be understood at this time that within the scope of the appended claims the invention may be practiced otherwise than as specifically described herein. It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “insetting an implant into a glenoid cavity” includes “instructing the insetting of an implant into the glenoid cavity.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10% =10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

1.-19. (canceled)

20. A humeral replacement prosthesis configured to be implanted into the humerus of a medical patient, comprising:
a proximal portion configured to support an articular surface;
a stem, extending at an angle with respect to a longitudinal axis of the proximal portion from the proximal portion to a distal end; and
three flanges extending radially outwardly from the longitudinal axis of the proximal portion and along a length of the proximal portion.

21. The humeral replacement prosthesis of claim 20, wherein the three flanges further extend distally towards the distal end of the stem.

22. The humeral replacement prosthesis of claim 20, wherein the three flanges extend distally to the distal end of the stem.

23. The humeral replacement prosthesis of claim 20, wherein the are integrally formed with the proximal portion.

24. The humeral replacement prosthesis of claim 20, wherein the three flanges are configured to support the articular surface.

25. The humeral replacement prosthesis of claim 20, wherein the flanges extend to a proximal end of the proximal portion

26. The humeral replacement prosthesis of claim 20, wherein central planes of the three flanges are circumferentially spaced from each other by about 120 degrees.

27. The humeral replacement prosthesis of claim 20, wherein a diameter of the stem at the distal end is about 5.5 mm or about 6 mm, or no more than about 7 mm, 6.5 mm, 6 mm, 5.5 mm, 5 mm, or less, or ranges including any two of the foregoing values.

28. The humeral replacement prosthesis of claim 20, wherein a diameter of the proximal portion is about 34 mm, 38 mm, or 44 mm, between about 25 mm and about 50 mm, such as about 25 mm, 30 mm, 35 mm, 40 mm, 45 mm, 50 mm, or is between 25 mm and 50 mm.

29. The humeral replacement prosthesis of claim 20, wherein a ratio of proximal-most diameter to distal-most diameter of the stem is between about 5 and about 9, between about 5.5 and about 8.5, about 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, or is between 4.5 and 10.

30. The humeral replacement prosthesis of claim 20, wherein the humeral replacement prosthesis has a total length of about or at least about 50 mm, 51 mm, 52 mm, 53 mm, 54 mm, 55 mm, 56 mm, 57 mm, 58 mm, 59 mm, 60 mm, 65 mm, 70 mm, or more, or between 50 mm and 70 mm.

31. The humeral replacement prosthesis of claim 20, wherein an external edge of each flange is curved along its length.

32. The humeral replacement prosthesis of claim 31, wherein the external edge of each flange is curved along its length from the articular surface to the stem.

33. The humeral replacement prosthesis of claim 20, wherein an external edge of each flange is curved along its width.

34. The humeral replacement prosthesis of claim 20, wherein the stem comprises at least three faces around a circumference of the stem.

35. The humeral replacement prosthesis of claim 20, wherein the proximal portion further comprises a proximal ring element.

36. The humeral replacement prosthesis of claim 35, further comprising at least one opening adjacent the proximal ring element.

37. The humeral replacement prosthesis of claim 20, further comprising the articular surface.

38. The humeral replacement prosthesis of claim 37, wherein the articular surface is convertible from anatomic to reverse.

39. The humeral replacement prosthesis of claim 20, wherein the proximal portion comprises an aperture to receive and align the articular surface.
